(19) United States
(12) Patent Application Publication
(10) Pub. No.: US 2025/0331885 A1
(43) Pub. Date: Oct. 30, 2025

(54) Punch Obturator

(71) Applicant: Arthrex, Inc., Naples, FL (US)

(72) Inventors: Nathanael I. GAMSO, Bonita Springs, FL (US); Kristin THORNE, Bonita Springs, FL (US); Matthew R. HERRINGTON, Naples, FL (US); Albert LIN, Naples, FL (US); Matthew T. PROVENCHER, Basalt, CO (US); James J. GUERRA, Naples, FL (US)

(73) Assignee: Arthrex, Inc., Naples, FL (US)

(21) Appl. No.: 19/189,554

(22) Filed: Apr. 25, 2025

Related U.S. Application Data
(60) Provisional application No. 63/638,450, filed on Apr. 25, 2024.

(51) Int. Cl. A61B 17/3205; A61B 17/04; A61B 17/16; A61B 17/34; A61F 2/08

(52) U.S. Cl. CPC A61B 17/32053; A61B 17/0401; A61B 17/1684; A61B 17/3421; A61B 17/3472; A61F 2/0811; A61B 2017/0409; A61B 2017/0464

(57) ABSTRACT

Surgical constructs, assemblies, kits and methods of tissue fixation are disclosed. A surgical instrument combines two separate devices into a single device. A surgical instrument combines a punch and a trocar/obturator into a single, integrated, combined device. A surgical punch obturator instrument can be a fixed length device. A surgical punch obturator instrument can be a spring-loaded device. A surgical punch obturator instrument can be a lever-arm device. A surgical punch obturator instrument can be provided with a soft stop.

### CROSS-REFERENCE TO RELATED APPLICATIONS

This application claims the benefit of U.S. Provisional Application No. 63/638,450 filed Apr. 25, 2024, the entire disclosure of which is incorporated by reference in its entirety herein.

### BACKGROUND

The present disclosure relates to the field of surgery and, more particularly, to surgical instruments and methods of surgical repairs.

### SUMMARY

Surgical instruments, assemblies, and methods are disclosed. A surgical instrument combines two separate devices into a single device. A surgical instrument combines a punch and a trocar/obturator into a single, integrated, combined device. A surgical instrument can be a spring-loaded device. A surgical instrument can be a lever-arm device. A surgical instrument can be provided with a soft stop.

Methods of surgeries are also disclosed. In an embodiment, a method of arthroscopic surgery is conducted with a combined surgical instrument which is configured to be employed both as a punch and a trocar/obturator.

### BRIEF DESCRIPTION OF THE DRAWINGS

FIGS. 1-5 illustrate various views of a punch obturator at different stages of actuation.

FIGS. 6-9 illustrate various views of another punch obturator at different stages of actuation.

FIGS. 10-15 illustrate various views of another punch obturator.

FIGS. 16 and 17 illustrate various views of another punch obturator.

FIGS. 18 and 19 illustrate various views of a surgical assembly with the punch obturator of FIGS. 16 and 17.

FIG. 20 illustrates a distal view of the punch obturator of FIG. 17.

FIG. 21 illustrates a view of a fixed punch obturator.

FIG. 22 illustrates an enlarged view of the distal end of the fixed punch obturator of FIG. 20.

FIGS. 23-26 illustrate various views of another punch obturator.

FIG. 27 illustrates an exemplary embodiment of a sleeve with soft stop.

FIG. 28 illustrates a punch obturator with the sleeve of FIG. 27.

FIGS. 29-31 illustrate exemplary embodiments of sleeves with soft stops.

FIGS. 32-34 illustrate various views of a spring punch sleeve with soft stop.

FIG. 35 illustrates a right-side view of the sleeve of FIG. 33.

FIGS. 36-38 illustrate various cross-sectional and partial cross-sectional views of punch obturator of FIG. 28.

FIGS. 39-41 illustrate various views of a wide footprint with cutout guide assembly.

FIG. 42 illustrates a right-side view of the assembly of FIG. 40.

FIGS. 43-46 illustrate various views of another wide footprint with cutout guide assembly.

FIG. 47 illustrates another exemplary punch obturator.

FIG. 48 illustrates another exemplary assembly with the punch obturator of FIG. 47.

FIG. 49 illustrates an enlarged view of the distal end of the punch obturator of FIG. 47.

FIGS. 50 and 51 illustrate views of a distal end of a tubing with laser lines.

FIGS. 52-54 illustrate steps of an exemplary method of reconstructive surgery with a punch obturator.

### DETAILED DESCRIPTION

The present disclosure provides surgical instruments, assemblies, kits, and methods of surgery.

A surgical instrument is both a punch and an obturator. A surgical instrument combines two separate devices (e.g., a punch and a trocar/obturator) into a single, integrated device. A punch obturator device can be provided with a soft stop.

A surgical instrument can be a spring-loaded device. A spring-loaded stop with a pin and slot in the handle allows movement of the punch-obturator shaft up and down to the same desired depths. A spring-loaded device can be provided with a soft stop. Although, in some implementations a soft stop may operate alone or without a spring mechanism.

A surgical instrument can be a lever-arm device. A lever in the form of a handle with rotating orientations allows for a shorter and longer depth stop. One length keeps the trocar shaft of the punch obturator at the tip of the drill guide, while the other length extends past the tip of the drill guide, to punch into bone at desired anchor depth. A lever-arm device can be provided with a soft stop.

Methods of endoscopic or arthroscopic surgeries are also disclosed. An exemplary method includes inter alia the step of employing a combined, integrated, single surgical instrument to conduct tissue punching and obturating with same instrument.

As detailed below, the instrument of the disclosure combines the function of a punch and trocar/obturator into a single device. With the device inserted into a guide, the device will stay in the trocar/obturator position to allow the user to advance the device through soft tissue to the anchor insertion site. The punch is malleted into bone, penetrating the cortex.

According to an exemplary embodiment, the combined punch obturator can include a spring-loaded stop with a pin and slot in the handle, to allow movement of the punch-obturator shaft up and down to the same desired depths.

According to another exemplary embodiment, the combined punch obturator can include a handle with rotating orientations to allow a shorter and longer depth stop. The handle of the punch can rotate to different orientations relative to the obturator to allow shorter and longer depth stops. One length keeps the trocar shaft of the punch obturator at the tip of the drill guide, while the other length can extend past the tip of the drill guide to punch into bone at typical anchor depth.

The embodiments provide an all-in-one design instead of being two separate devices.

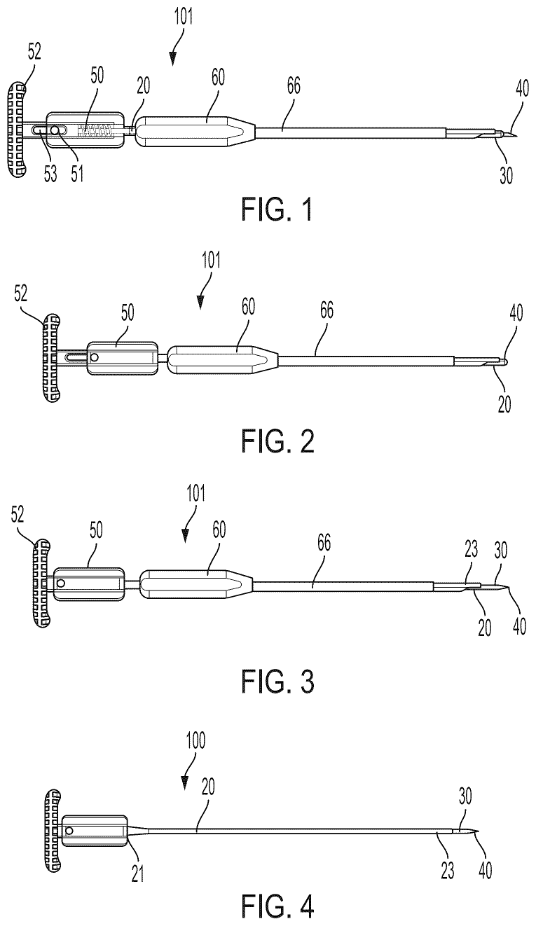

Referring now to the drawings, where like elements are designated by like reference numerals, FIGS. 1-51 illustrate various views of exemplary punch obturator 100, 200, 300 . . . 800 (punch trocar/obturator 100, 200, 300 . . . 800; punch trocar 100, 200, 300 . . . 800; surgical instrument 100, 200, 300 . . . 800; device 100, 200, 300 . . . 800) and associated surgical assemblies. FIGS. 52-54 illustrate steps of exemplary method of reconstructive surgery with punch obturator 100, 200, 300 . . . 800 of the present disclosure.

Punch obturator 100, 200, 300 . . . 800 can be employed, for example, as part of a surgical procedure directed at repairing soft tissue injuries (e.g., rotator cuff injuries). To that end, punch obturator 100, 200, 300 . . . 800 is configured for advancing through soft tissue and for penetrating and creating an opening (or pilot hole) in a hard surface (e.g., a bony targeted location) beneath the soft tissue. This opening can then be utilized for receiving and anchoring a fixation device, such as a suture anchor, for use in repairing the soft tissue injury. Punch obturator 100, 200, 300 . . . 800 can be used alone, or in combination with a drill and cannula guide as part of a punch obturator assembly.

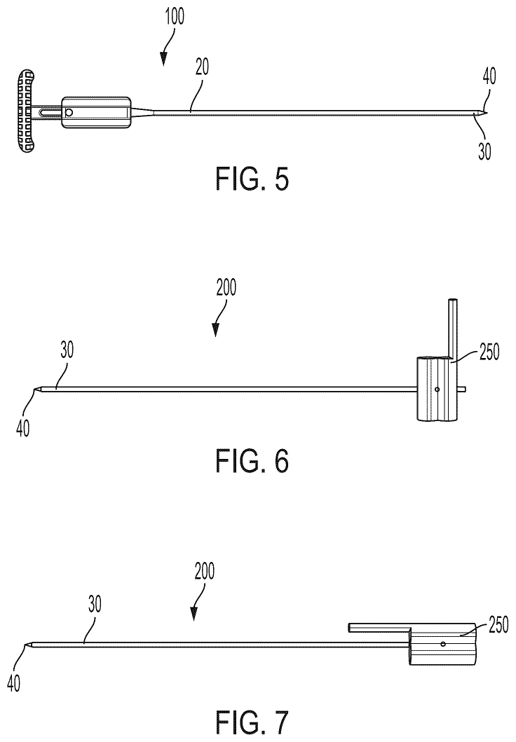

FIGS. 1-5 illustrate various views of an exemplary punch obturator 100. Punch obturator 100 can be part of surgical assembly 101 (shown in FIGS. 1-3) which includes punch obturator 100 secured to drill guide 60 and slidable within drill guide cannula 66. FIGS. 4 and 5 depict punch obturator 100 out of the assembly 101, such as out of the drill guide 60. As detailed below, punch obturator 100 of FIG. 1 is in neutral, normal position. Punch obturator 100 is in an extended position in FIG. 3.

Punch obturator 100 may be a spring-loaded device. A spring-loaded stop with a pin and slot in the handle allows movement of the punch-obturator shaft up and down to the same desired depths.

FIG. 1 shows surgical assembly 101 with punch obturator 100 comprising handle 52, spring 50, and pin and slot mechanism 51, 53 (pin 51 and slot 53). Handle 52 can be a transparent handle (as shown in FIG. 1) to show the spring 50 inside the device 100. Punch obturator 100 is slidably and removably located within drill guide 60 and drill guide cannula 66.

Punch obturator 100 also may include a tube, sleeve, or cannula 20 (shown in FIGS. 4 and 5) that has a hollow, elongated body with a proximal end 21 (attached to handle 52) and an open distal end 23. The cannula body can be rigid or partially rigid and can be formed of various materials such as metals, alloys, polymers, plastics, or combinations thereof. The tube, sleeve, or cannula 20 can have various cross-sections such as circular or elliptical, among many others. The cannula body can be constructed with enough strength and rigidity to withstand being forcibly advanced through soft tissue and/or bone, such as with a mallet or similar device, during a surgical procedure.

During use, punch obturator 100 can be inserted through a drill guide 60 and used with the drill guide, as detailed below. If the tube/sleeve 20 is formed of metal or similar materials such as metal alloys, the punch obturator 100 can be used independently, for example, without a drill guide. In an embodiment, sleeve 20 can be a silicone-like sleeve (or a sleeve formed of softer materials such as plastics) which can require the use of a drill guide for additional stability. However, punch obturator 100 may be also employed without a drill guide.

A shaft 30 (e.g., a punch obturator shaft) is provided at its most distal end with a perforating tip 40 (tip 40; sharp point 40). In some implementations described throughout, the shaft 30 may comprise a metal shaft and in some implementations the shaft 30 may comprise a medical plastic. Shaft 30 is housed within tube 20 and is configured to move up and down the tube 20 by actuating handle 52 and spring 50. FIG. 1 shows the punch obturator 100 in an intermediate (normal) position, for example, an about half-pressed (half-compressed) position wherein shaft 30 with sharp point 40 only partially extend beyond cannula 66 (beyond most distal end of cannula 66).

FIG. 2 shows the punch obturator 100 in neutral obturator position (a first position; one exemplary position; obturator position; non-pressed position). Tube or cannula 20 can be a plastic sleeve attached to the handle and protrudes out of drill guide cannula 66. The tube 20 covers the punch so that shaft 30 is located inside of plastic sleeve, to fill cannula 66 and prevent soft tissue from entering shaft. Shaft 30 can be a metal shaft. In this position, device 100 acts as a trocar/obturator. Device 100 inserted into guide 60 remains in the trocar/obturator position to allow the user to advance the device through skin and soft tissue (with sharp tip 40 and cannula 20 penetrating skin and soft tissue) and be positioned in the vicinity of hard tissue such as bone (at the anchor insertion site).

FIG. 3 shows the punch obturator 100 in a pressed punch position (a second position; another exemplary position; pressed position; fully pressed position). With the spring 50 fully compressed, the metal punch shaft 30 is protruding out of the entire assembly 101, to allow the sharp point 40 of the punch to break bone cortex and form a pilot hole to allow for subsequent fixation device insertion (for example, anchor insertion).

The spring-loaded stop with a pin and slot 51, 53 in the handle 52 and spring 50 allow movement of the punch-obturator shaft 30 up and down to the same desired depths. As such, and for a better understanding, FIG. 4 illustrates the device 100 out of the drill guide 60 and in the punch position. FIG. 5 illustrates the device 100 out of the drill guide 60 and in obturator position. In the obturator position, the shaft 30 and tip 40 are about fully covered by and located within sleeve 20. Sharp tip 40 can extend/protrude out of the sleeve 20 by about 1-5 mm, preferably about 4-5 mm, to help penetrate tissue.

FIGS. 6-9 illustrate various views of exemplary punch obturator 200. Punch obturator 200 is about similar to punch obturator 100 detailed above but differs in that the spring-loaded mechanism 50, 51, 53 of punch obturator 100 is replaced by a handle with rotating orientations to allow a shorter and longer depth stop. One length keeps the trocar shaft of the punch obturator at the tip of the drill guide, while the other length can extend past the tip of the drill guide to punch into bone at typical anchor depth.

Punch obturator 200 may comprise a lever-arm device. A lever 250 in the form of a handle with rotating orientations allows for a shorter and longer depth stop. The lever 250 may rotate about an axis perpendicular to the longitudinal axis of the shaft 30. One length keeps the shaft 30 of the punch obturator 200 at the tip of the drill guide 60, while the other length extends past the tip of the drill guide, to punch into bone at typical anchor depth.

FIG. 6 illustrates punch obturator 200 (the lever-arm device 200) in punch position and without the drill guide 60. At this position, lever 250 is oriented about perpendicular to a longitudinal axis of the instrument.

FIG. 7 illustrates punch obturator 200 in obturator position and without the drill guide 60. At this position, lever 250 is oriented about parallel to a longitudinal axis of the instrument.

FIG. 8 illustrates punch obturator 200 in punch position, with the metal shaft protruding out of cannula 66 and as part of surgical assembly 201.

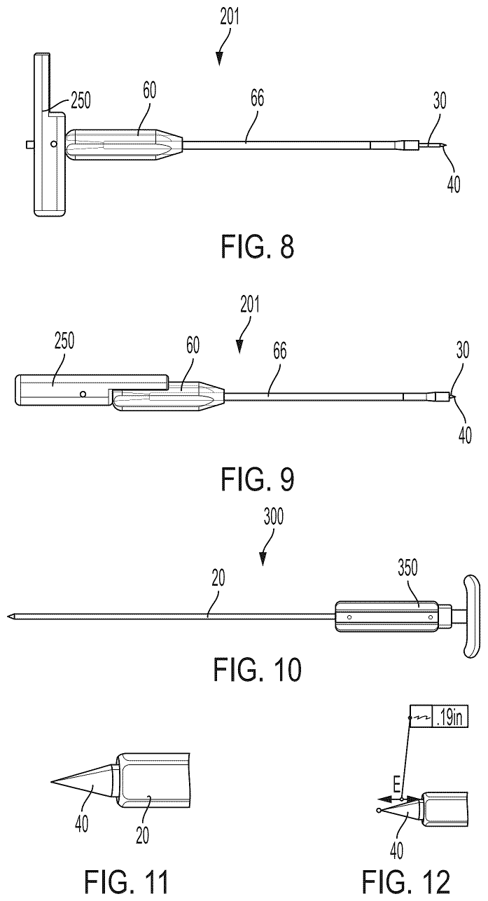

FIG. 9 illustrates punch obturator 200 in obturator position and as part of surgical assembly 201. The shaft 30 is partially sticking out of drill guide shaft to prevent tissue from entering drill guide cannula 66.

FIGS. 10-15 illustrate various views of another exemplary punch obturator 300. Punch obturator 300 is about similar to punch obturator 100 detailed above but differs in that handle 350 (shown in detail in FIG. 14) is provided with a stop feature 355 that offers the surgeon tactile feedback when the surgeon has depressed handle 350 to decrease over-punching. Stop feature 355 can be in the form of an o-ring, such as a silicone o-ring, for example, or similar structure. Stop feature 355 can be a “soft stop” formed of a soft material that can provide a degree of flexibility to stop feature 355. In certain implementations, the soft stop may be formed of foam, rubber, elastomers, or similar materials, or combinations thereof. In certain implementations, the soft stop may comprise foam, rubber, elastomers, or combinations thereof. In certain implementations, the soft stop may be formed of any medical grade plastic or polymeric material that can confer flexibility. In certain implementations, the soft stop may comprise PEBA material (Polyether Block Amide material) which is a type of thermoplastic elastomer (TPE) that shares similarities with other TPEs like Thermoplastic Polyurethanes (TPU) and polyester elastomers. In additional implementations, the soft stop may include EVA foam (Ethylene-Vinyl Acetate foam). In yet additional implementations, the soft stop may comprise (or consist essentially of) any combination of PEBA, TPE, and/or EVA materials.

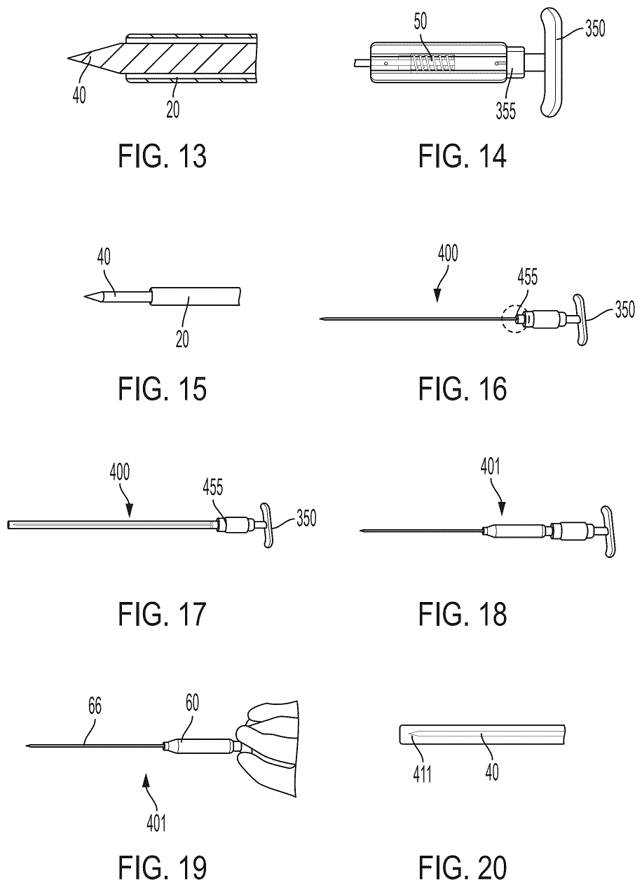

FIGS. 11 and 12 illustrate side views of the distal end (tip) of the punch obturator 300. FIG. 13 illustrates a longitudinal, cross-sectional view of the distal end (tip) of FIGS. 11 and 12. In FIGS. 11-13, the punch obturator 300 is in obturator position and without the drill guide 60. In certain implementations, distal tip 40 may extend outside the sleeve. In certain implementations, distal tip 40 may extend outside sleeve 20 by a distance “E” (FIG. 12). In certain implementations, distance E may be of about 0.5-5 mm, more preferably about 4-5 mm. FIG. 14 illustrates an enlarged view of handle 350 with soft stop 355 detailed above located between spring mechanism 50 and handle 350. Soft stop 355 abuts the spring housing and/or interacts with the spring mechanism.

FIG. 15 illustrates punch obturator 300 in punch position and without the drill guide 60. In the punch position, the tip may extend out of the sleeve 20 and drill guide shaft 66. In certain implementations, the tip may extend for about 5-12 mm, more preferably, for about 10-12 mm.

Reference is now made to FIGS. 16-20 which illustrate exemplary embodiment 400. Punch obturator 400 is about similar to punch obturator 300 detailed above but differs in that punch obturator 400 is not a spring-loaded device but rather a fixed device, a T-handle punch obturator. In addition, the soft stop in punch obturator 400 is positioned between the guide and the punch (as opposed to between the T-handle and the spring housing). Stop feature 455 may be similar to (and may be formed of a material similar to that of) stop feature 355 described above. Stop feature 455 may be positioned outside the spring housing. Stop feature 455 may be positioned on either side of the spring housing. In certain implementations, stop feature 455 may be located within or partially within the spring housing. Punch obturator 400 includes a tube 411 (FIGS. 17 and 20) over the needle 40 so this could function as an obturator. This device may fit down the guide 60 until the guide hits the soft stop 455. The device may then be malleted punching to a depth of about 0.5 mm to about 18 mm. FIGS. 18 and 19 illustrate assembly 401 including punch obturator 400 with exemplary drill guide 60 and cannula 66. The soft stop acts as a cushion/grommet to back of the device so the surgeon knows when to stop when malletting. In certain implementations, handle 350 can be plastic molded so the guide could nest into it.

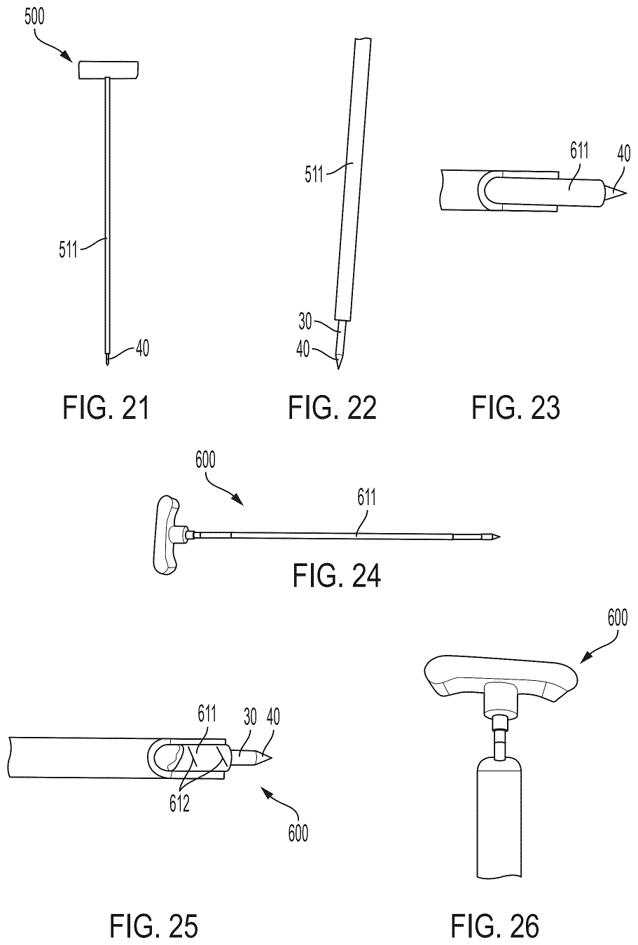

FIGS. 21 and 22 illustrate various views of an exemplary punch 500. Punch 500 is about similar to punch obturator 400 detailed above but differs in that punch 500 is a fixed punch without a soft stop, such as soft stop 455. Punch 500 is also not provided with a spring. In this implementation, the tubing 511 is over the shaft 30, and may expose tip 40.

Punch obturator 600 of FIGS. 23-26 incorporates the soft stop concept into the tubing. Tubing 611 may be provided over the shaft 30 and tip40. Tubing 611 may act as a soft stop (similar to soft stops 355, 455 detailed above) since it may incorporate a soft stop material. In this exemplary embodiment, tubing 611 itself is a soft stop. As detailed above, a soft material for tubing 611 (soft stop 611) may be any material that can provide a degree of flexibility to tubing 611. In certain implementations, tubing 611 may be formed of foam, rubber, elastomers, or similar materials, or combinations thereof. In certain implementations tubing 611 may be formed of any medical grade plastic or polymeric material that can confer flexibility. In certain implementations, tubing 611 may comprise PEBA material (Polyether Block Amide material) which is a type of thermoplastic elastomer (TPE) that shares similarities with other TPEs like Thermoplastic Polyurethanes (TPU) and polyester elastomers. In additional implementations, tubing 611may include EVA foam (Ethylene-Vinyl Acetate foam). In yet additional implementations, tubing 611 may comprise (or consist essentially) of any combination of PEBA, TPE, and/or EVA materials. A cut 612 (e.g., a spiral cut) in tubing shown in FIG. 25 and/or the addition of a spring may add to the soft stop feature. Tubing 611 can be cut or can be molded with openings (e.g. in the side wall) along the length of the tubing. In certain implementations, cuts may be provided along a longitudinal axis of the tubing. In certain implementations, one or more cuts along the longitudinal axis of the tubing can allow the tubing to buckle and/or bow and/or fold or bend at the surgical site, and these cuts act as additional stop features. As detailed below, the one or more cuts may be provided at any location along the length of the tubing 611 and may be provided in a pattern or regular arrangement, or in a non-regular arrangement or pattern. Tubing 611 may also include lines (e.g., laser lines 612 depicted in black) for aligning with lines (e.g., laser lines) on the guide, and as detailed below.

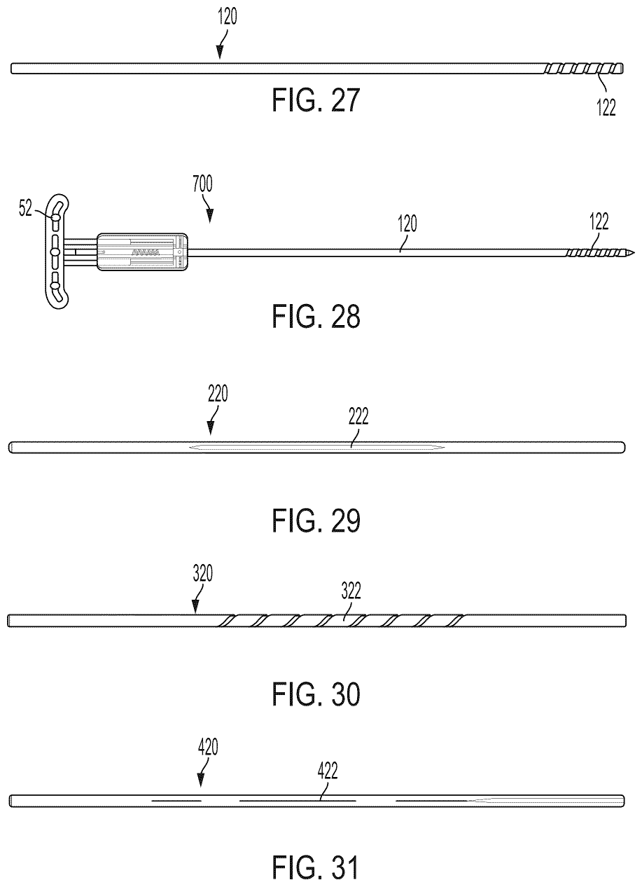

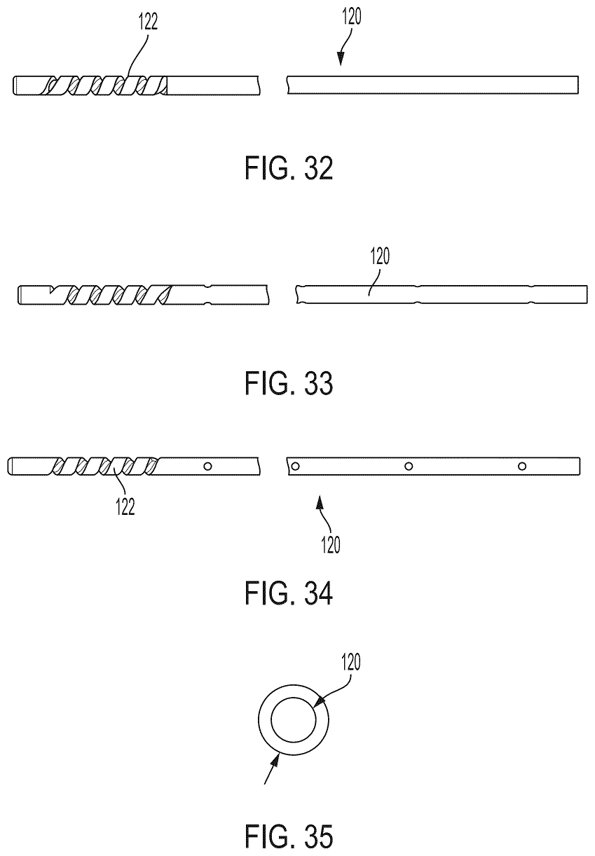

Reference is now made to FIGS. 27-49 which illustrate various views of exemplary punch obturator 700, 800 and assembly 801. Punch obturator 700 is about similar to punch obturator 100 detailed above in that it also includes a spring mechanism; however, punch obturator 700 may be provided with either a sleeve with spiral cuts and/or flared design or with a tube 20 provided with spiral cuts. For example, FIG. 27 shows sleeve 120 having spiral cut 122 that acts as a soft stop and part of punch obturator 700 of FIG. 28. FIGS. 29-31 illustrate sleeves 220, 320, 420 with exemplary cuts/pattern/indentations 222, 322, 422. Exemplary cut 222 of FIG. 29 is a singular longitudinal cut provided along a length of sleeve 220. Exemplary cuts 422 of FIG. 31 include a plurality of longitudinal multiple cuts provided along a length of sleeve 420. Exemplary spiral cut 322 of FIG. 30 is provided at a location about the mid-length of sleeve 320. FIGS. 32-35 illustrate various views and additional details of spring punch sleeve 120 with exemplary soft stop 122. Exemplary cuts/pattern/indentations 122, 222, 322, 422 can be provided anywhere along the length of the tubing/sleeve to allow the tubing/sleeve to buckle (bow, fold, collapse, bend) at the surgical site. Exemplary cuts/pattern/indentations 122, 222, 322, 422 can be provided in any pattern and any combination along the tubing/sleeve. Exemplary cuts/pattern/indentations 122, 222, 322, 422 can be provided as additional soft features to the tubing/sleeve that acts as a soft stop or, alternatively, cuts/pattern/indentations 122, 222, 322, 422 can be provided to act as the soft stop.

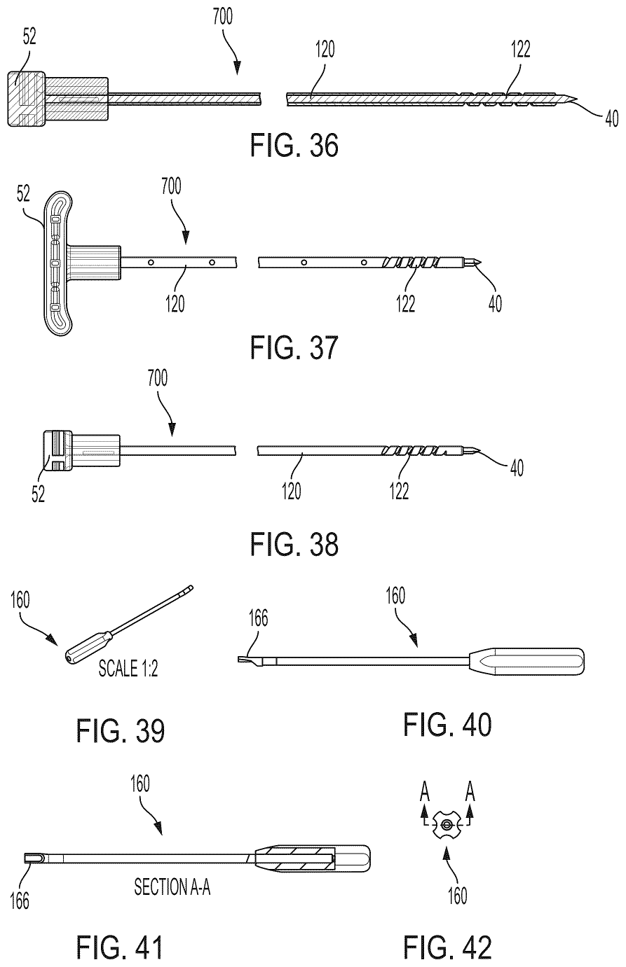

FIGS. 36-38 illustrate various cross-sectional views of trocar punch 700 of FIG. 28. Punch shaft 120 is provided with spiral cut 122.

FIGS. 39-42 illustrate various views of a wide footprint with cutout guide assembly 160 that may be disposable. Wide footprint 166 is provided at a most distal end of guide 160.

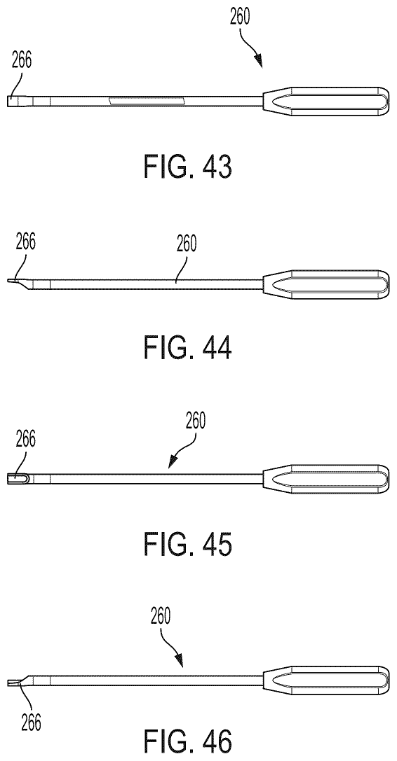

FIGS. 43-46 depict various views of a reusable wide footprint with cutout guide 260. Wide footprint 266 is provided at a most distal end of guide 260.

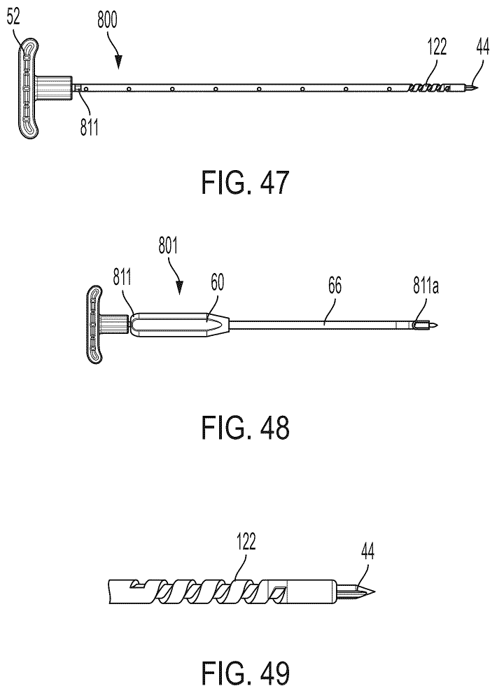

Reference is now made to FIGS. 47-49 which illustrate various views of another exemplary punch obturator 800 and assembly 801. Punch obturator 800 is provided with one or more laser lines 811 adjacent to handle 52, a cutout for visual depth stop 122, and a cutting flute 44.

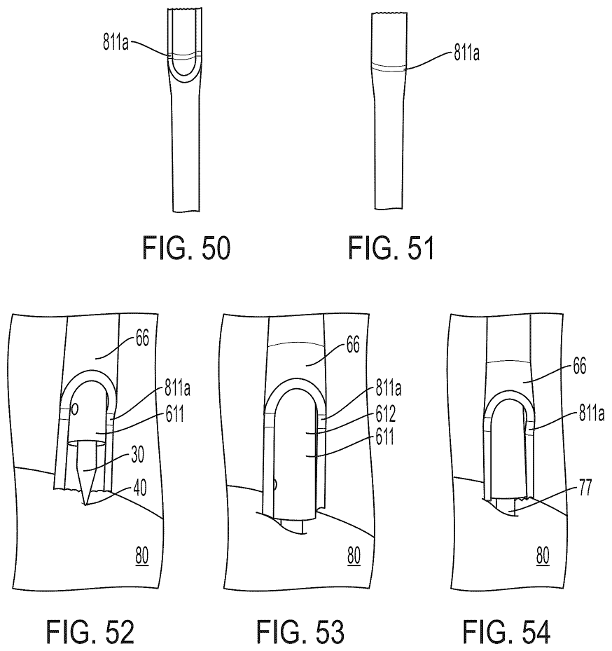

In assembly 801 of FIG. 48, multiple laser lines are provided. Laser lines may be located on both proximal and distal ends: laser line on guide that matches distal end, and proximal laser line that shows when to start slowing down. Laser line 811 bottoms out and laser line 811a appears when the sleeve meets the cortex. An enlarged depiction of most distal end of the punch obturator 800 is shown in FIG. 49. FIGS. 50 and 51 illustrate enlarged views of a tubing with laser lines 811a of FIG. 48.

Punch obturator 100, 200, 300 . . . 800 combines the function of a punch and trocar/obturator into a single, integrated instrument. With the device 100, 200, 300 . . . 800 inserted into a guide 60, 66, the punch obturator 100, 200, 300 . . . 800 can stay in the trocar/obturator position to allow the user to advance the device through soft tissue at the anchor insertion site. The punch obturator 100, 200, 300 . . . 800 can then be actuated into the punch position to allow the punch to be malleted into bone, breaking the cortex. The punch can create a pilot hole at a bone location corresponding to the insertion site of a fixation device (for example, an anchor). If necessary, a drill and/or cutting instrument can create a bone hole for anchor insertion. The drill/cutting instrument can be inserted through drill guide 60 or through a similar device.

Punch obturators 100, 200, 300 . . . 800 detailed above are surgical instruments with applicability to various open or arthroscopic procedures including procedures for re-approximating bone to bone or soft tissue to bone, for example, shoulder rotator cuff repairs, capsulolabral reconstruction, SLAP repairs, as well as ankle, knee, elbow or foot repairs, among many others.

Reference is now made to FIGS. 52-54, which illustrate a partial distal view of a repair site (exemplary shoulder repair site such as a rotator cuff repair) with surgical assembly and exemplary punch obturator 100, 200, 300 . . . 800 passed through a percutaneous incision and placed onto the desired anchor insertion site on humerus 80. Punch obturator 100, 200, 300 . . . 800 can be employed to form at least one pilot hole within the humeral tuberosity. Additional cutting devices can be employed, if necessary and/or desired, to form one or more bone holes to allow subsequent insertion and fixation of at least one fixation device.

FIG. 52: Guide 66 is lined up against the cortex so both the punch and the guide are against bone 80.

FIG. 53: Punch is malleted until laser line 612 matches laser line 811a on guide.

FIG. 54: Fixation device (anchor) 77 is inserted until laser line matches with laser line on guide.

A plurality of pilot holes may be formed with punch obturator 100, 200, 300 . . . 800 to allow insertion of one or more fixation devices 77 with associated sutures (flexible strands) for attachment of the rotator cuff to glenoid as part of a shoulder repair.

Fixation devices 77 can be anchors, for example, knotted anchors, knotless anchors, or all-suture anchors, or any devices that confer secure attachment and fixation of soft tissue over bone. The fixation device 77 can be a knotless anchor such as a two-piece Arthrex PushLock® anchor, disclosed in U.S. Pat. No. 7,329,272, or an Arthrex SwiveLock® anchor, disclosed in U.S. Pat. Nos. 8,012,174 and 9,005,246, the disclosures of both of which are fully incorporated by reference in their entirety herein.

Fixation device 77 can be an all-suture soft anchor (soft suture anchor) provided with a soft anchor sleeve (sheath, tubular member) with two open ends and one or more flexible shuttling strands extending through the soft anchor sleeve (sheath). Flexible strands can extend through the sleeve in similar or different directions and/or orientations and/or locations. The flexible sleeve with the one or more shuttling strands is secured into or onto bone, and the strands allow passing of additional flexible strands such as sutures or tapes to pass over soft tissue and be secured into bone to approximate soft tissue to bone. Details of an exemplary soft suture anchor with a soft anchor sleeve (sheath or tubular member) and flexible shuttling strands are set forth, for example, in U.S. Pat. No. 10,849,734 issued Dec. 1, 2020, entitled “Methods of Tissue Repairs,” the disclosure of which is incorporated by reference in its entirety herein.

If a soft anchor is employed, the flexible coupler can be used for attachment of soft tissue to bone by passing the flexible coupler through or around soft tissue and forming at least one adjustable, flexible, continuous, knotless, tensionable loop around or through soft tissue. One or more flexible couplers and optional shuttling strands can extend through the lumen of sleeve in similar or different directions and/or orientations and/or locations. The flexible tubular sleeve of fixation device 77 with the flexible couplers and shuttling strands may be secured into or onto bone (for example, into a pilot hole formed with punch obturator 100, 200, 300 . . . 800), and flexible strands can pass over soft tissue (rotator cuff) and are secured into bone to approximate soft tissue to bone.

A punch obturator 100, 200, 300 . . . 800 comprises: a tube or cannula 20 having a proximal end 21 and a distal end 23; a handle 52 attached to the proximal end 21 of the tube or cannula 20; and a shaft 30 attached to the handle 52 and slidably located within the tube or cannula 20. The shaft can further include a most distal sharp tip 40 for penetrating tissue. A punch obturator can be a spring-loaded device. A punch obturator can be a lever-handle device. A punch obturator can be provided with one or more stop features to provide tactile feedback to surgeon. A punch obturator 100, 200, 300 . . . 800 can be an integrated device that can be used as both a trocar/obturator and a punch. A punch obturator 100, 200, 300 . . . 800 can include a handle; a shaft attached to the handle; and an integrated soft stop. A soft stop can include foam, rubber, elastomers, or similar materials, or combinations thereof. A soft stop can be formed of any medical grade plastic or polymeric material that can confer flexibility. A soft stop can be formed of PEBA (Polyether Block Amide) TPU (Thermoplastic Polyurethanes), EVA foam (Ethylene-Vinyl Acetate foam), or combinations thereof.

A punch obturator assembly comprises: a drill guide 60 with drill guide cannula 66; and a punch obturator 100, 200, 300 . . . 800 slidably and removably attached to the drill guide 60. The punch obturator 100, 200, 300 . . . 800 can be a spring-loaded device or a lever-handle device. Actuation of handle 52 and compression of spring 50 allow the shaft 30 to protrude out of the most distal end 23 of the tube or cannula 20, into a punch position. The handle can be actuated by compressing (pressing on) spring 50 or by rotating lever 250 from a first position (about parallel to the longitudinal axis of the device) to a second position (about perpendicular to the longitudinal axis of the device).

Punch obturator 100, 200, 300 . . . 800 described above can be included in a surgical kit, assembly, or system to simplify the surgeon's task of selecting a specific instrument and to aid in the overall surgical procedure. A surgical kit for an arthroscopic surgical repair can include one or more punch obturators 100, 200, 300 . . . 800; assemblies 101, 201 . . . 801; awls or equivalent devices, as well as guide drills, and/or drills, and/or or bone-penetrating devices such as various cutting devices. The surgical kit can also include fixation devices (such as anchors 77, screws, suture-button constructs, plates, etc.) and flexible members (such as sutures) to be employed in conjunction with the bone tunnels, bores or holes formed with punch obturator 100, 200, 300 . . . 800.

Methods of endoscopic or arthroscopic surgeries are also disclosed. An exemplary method includes inter alia the steps of: (i) inserting a punch obturator 100, 200, 300 . . . 800 through skin and soft tissue; (ii) forming at least one pilot hole within bone with the punch obturator 100, 200, 300 . . . 800; and (iii) attaching soft tissue to the bone. The method can further include the steps of: passing at least one fixation device through the drill guide 60 and cannula 66; and securing the at least one fixation device 77 into the pilot hole. The method can further include the steps of: optionally, drilling a hole into bone at the at least one pilot hole location; and securing the soft tissue to the bone with a plurality of fixation devices 77 and flexible strands. At least one of the plurality of fixation devices can be an all-suture soft anchor secured into the hole. The bone can be the glenoid and soft tissue can be rotator cuff.

The flexible strands can be formed of any flexible material such as suture or tape, or combination of suture and tape. The suture can be in the form of any known suture construct, such as multifilament, braided, knitted, woven suture, or including fibers of ultrahigh molecular weight polyethylene (UHMWPE) or the FiberWire® suture (disclosed in U.S. Pat. No. 6,716,234, the disclosure of which is hereby incorporated by reference in its entirety herein). The tape can be formed of suture tape, for example, Arthrex FiberTape®, which is a high strength suture tape that is braided and rectangular-like in cross section and as disclosed in U.S. Pat. No. 7,892,256, the disclosure of which is hereby incorporated by reference in its entirety herein. Flexible strand can include any soft, flexible strand of material. Flexible strand can consist essentially of elastic suture.

The surgical constructs of the present disclosure may be employed in endoscopic surgery. The term “endoscopic surgery” refers to surgical procedures within a patient's body through small openings as opposed to conventional open surgery through large incisions. Additionally, surgical constructs as disclosed herein may be utilized in other general surgical and specialty procedures such as soft tissue repairs.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed is:

1. A punch obturator comprising:
a handle;
a shaft attached to the handle and configured to extend between an obturator position and a punch position; and
a tubing housing the shaft.

2. The punch obturator of claim 1, wherein the tubing is configured to act as a soft stop.

3. The punch obturator of claim 2, wherein the tubing is provided with at least one cut or opening.

4. The punch obturator of claim 3, wherein the tubing is provided with a plurality of cuts or openings forming a spiral around the shaft.

5. The punch obturator of claim 3, wherein the tubing is provided with a plurality of cuts or openings extending about parallel to a longitudinal axis of the tubing.

6. The punch obturator of claim 5, wherein the tubing is provided with a plurality of cuts or openings forming a regular pattern.

7. The punch obturator of claim 5, wherein the tubing is provided with a plurality of cuts or openings forming an irregular pattern.

8. The punch obturator of claim 1, wherein the shaft is formed of metal.

9. The punch obturator of claim 1, wherein the tubing is formed of plastic.

10. The punch obturator of claim 1, wherein the tubing is formed of foam, rubber, elastomers, polymers or combinations thereof.

11. The punch obturator of claim 10, wherein the tubing is formed of Polyether Block Amide material.

12. The punch obturator of claim 1, wherein, when the shaft is in the obturator position, the shaft is located about fully within the tubing.

13. The punch obturator of claim 1, wherein, when the shaft is in the obturator position, the shaft protrudes out of the tubing for about 1 to about 5 mm to help penetrate tissue.

14. The punch obturator of claim 1, wherein the handle is a spring actuated handle.

15. The punch obturator of claim 1, wherein the handle is a lever configured to rotate for about 90 degrees relative to a longitudinal axis of the shaft.

16. The punch obturator of claim 1, wherein the punch obturator is employed in arthroscopic shoulder surgery.

17. A surgical kit, comprising:
a punch obturator as in claim 1; and
at least one fixation device.

18. The surgical kit of claim 17, further comprising a drill guide.

19. The surgical kit of claim 17, wherein the at least one fixation device is a soft suture anchor comprising a flexible tubular sleeve or sheath and a plurality of flexible strands extending through a passage of the flexible tubular sleeve or sheath.

20. A method of tissue repair, comprising:
piercing at least one of skin and soft tissue with a punch obturator; and
forming a pilot hole in bone with the punch obturator.

21. The method of claim 20, further comprising actuating a handle of the punch obturator from a first position to a second position.

22. The method of claim 21, wherein the first position is an obturator position and wherein the second position is a punch position.

23. The method of claim 20, further comprising:
securing at least one fixation device into the pilot hole; and
securing soft tissue to the bone by employing at least one flexible strand attached to the at least one fixation device.

24. The method of claim 20, wherein the tissue repair is rotator cuff repair.
